(19) United States
(12) Patent Application Publication
(10) Pub. No.: US 2006/0247549 A1
(43) Pub. Date: Nov. 2, 2006

(54) Wireless heart rate monitoring system

(75) Inventor: Raymond Chan, Hong Kong (HK)

Correspondence Address: LEYDIG VOIT & MAYER, LTD, 700 THIRTEENTH ST. NW, SUITE 300, WASHINGTON, DC, 20005-3960, US

(73) Assignee: IDT Technology Limited, Hunghom (HK)

(21) Appl. No.: 11/117,484

(22) Filed: Apr. 29, 2005

(51) Int. Cl. A61B 5/04

(52) U.S. Cl. 600/519

(57) ABSTRACT

A wireless heart rate monitoring system comprises a detection unit and a display unit, the two units being separate and co-operatively in communication via a wireless link. The detection unit comprises a casing, straps for attaching the casing on a user, a heart rate detector at the casing for detecting ECG pulses of the user, a microprocessor for computing a heart rate based on the ECG pulses detected by the heart rate detector, and a transmitter for transmitting a wireless signal indicative of the computed heart rate to the display unit. The display unit comprises a casing, a receiver for receiving the wireless signal from the detection unit, and a display for displaying the heart rate received by the receiver.

The present invention relates to a heart rate monitor.

### BACKGROUND OF THE INVENTION

More particularly, although not exclusively, the invention is concerned with a wireless-transmission heart rate monitor comprising a body-mounted heart beat detector and a remote receiver/display unit which might also be worn on the body, for example like a watch about the wearer's wrist.

Conventional heart rate monitors include a body-mounted detector such as a chest belt which transmits a signal to the remote display unit upon detection of each heart beat. The remote display unit receives such signals and then calculates an ECG (electrocardiogram) pulse count for display purposes. Such apparatus are generally reliable in environments free of interference that may be caused by radiation from nearby transmitting devices such as mobile phones or power lines. This problem has been addressed to in U.S. Pat. Nos. 5,611,346 and 5,632,279.

In the former document, two identification pulses are generated by each body-mounted transmitter whereby the timing of all between the identification pulses corresponds to a specific time interval determined to reach transmitter-receiver pair, on the basis of which specific interval the receiver identifies the measuring pulses intended for it. In the latter document, heart beat signals (bursts) are amplitude-modulated with a frequency or a frequency sequence characteristic of the transmitter/receiver unit, on the basis of which the receiver recognises the bursts only intended for it.

In the known prior art, heart rate monitors including those disclosed in the above referenced US patents, the actual heart rate data (typically in beats per minute) to be displayed is not computed within the body-mounted detector unit, but rather at a remote display location for example by a wrist-watch-type main unit worn on the wrist. Moreover, the wireless signals transmitted by the transmitter to the receiver are in the form of ECG bursts to be computed at the receiving end of the wireless transmission so to speak. Accordingly, complex and often unreliable methods must be employed in an attempt to distinguish one transmission from another where multiple users—say in a hospital or sporting environment—are wearing body-mounted transmitters in local proximity to one another.

### OBJECTS OF THE INVENTION

It is an object of the present invention to overcome or substantially ameliorate the above disadvantages and/or more generally to provide an improved wireless heart rate monitor.

### SUMMARY OF THE INVENTION

According to the invention, there is provided a wireless heart rate monitoring system comprising a detection unit and a display unit, the two units being separate and co-operatively in communication via a wireless link. The detection unit comprises a casing, attaching means at the casing for attaching the casing on a user, a heart rate detector at the casing for detecting ECG pulses of said user, a microprocessor in the casing for computing a heart rate based on the ECG pulses detected by the heart rate detector, and a transmitter at the casing for transmitting a wireless signal indicative of the heart rate computed by the microprocessor to the display unit. The display unit comprises a casing, a receiver at the casing for receiving said wireless signal from the detection unit, and a display at the casing for displaying the heart rate received by the receiver.

Preferably, the attaching means comprises a strap or straps for attaching around the chest of said user.

Preferably, the display unit is of a wrist-mounted type.

It is preferred that the transmitter comprises a radio frequency transmitter and the receiver comprises a radio frequency receiver.

It is preferred that the transmitter is controlled by the microprocessor to transmit said wireless signal at substantially fixed intervals.

In a preferred embodiment, the detection unit includes timing means for controlling its transmitter to transmit said wireless signal at a different time from that of another detection unit nearby.

More preferably, the transmitter is controlled by the microprocessor to transmit said wireless signal at substantially fixed intervals, and the timing means comprises a series of time slots divided from said fixed interval, one of which time slots is allocated for the transmitter to transmit said wireless signal.

Further more preferably, the detection unit includes an operator, successive depression of which shifts the time of transmission of said wireless signal from one time slot to the next until a vacant time slot is reached.

In a preferred embodiment, said wireless signal comprises a coded data packet that includes data representing the heart rate computed by the microprocessor.

More preferably, the coded data packet further includes a code identifying the detection unit.

### BRIEF DESCRIPTION OF DRAWINGS

The invention will now be more particularly described, by way of example only, with reference to the accompanying drawings, in which:

FIG. 1 is a front view of an embodiment of a wireless heart rate monitoring system in accordance with the invention, comprising a chest-belt detection unit and a wrist-mounted display unit;

FIG. 2 is a schematic functional block diagram of the transmitting and receiving units of FIG. 1;

FIGS. 3A to 3C are schematic waveform diagrams showing comparison of transmission protocol between the present invention and the prior art; and

FIGS. 4A to 4C are schematic waveform diagrams showing signals transmitted by the detection unit to the-display unit.

### DETAILED DESCRIPTION OF PREFERRED EMBODIMENT

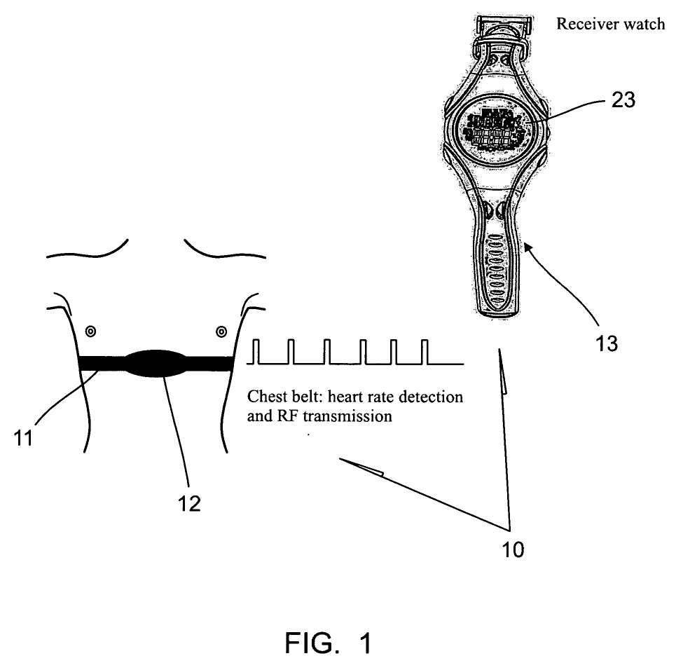

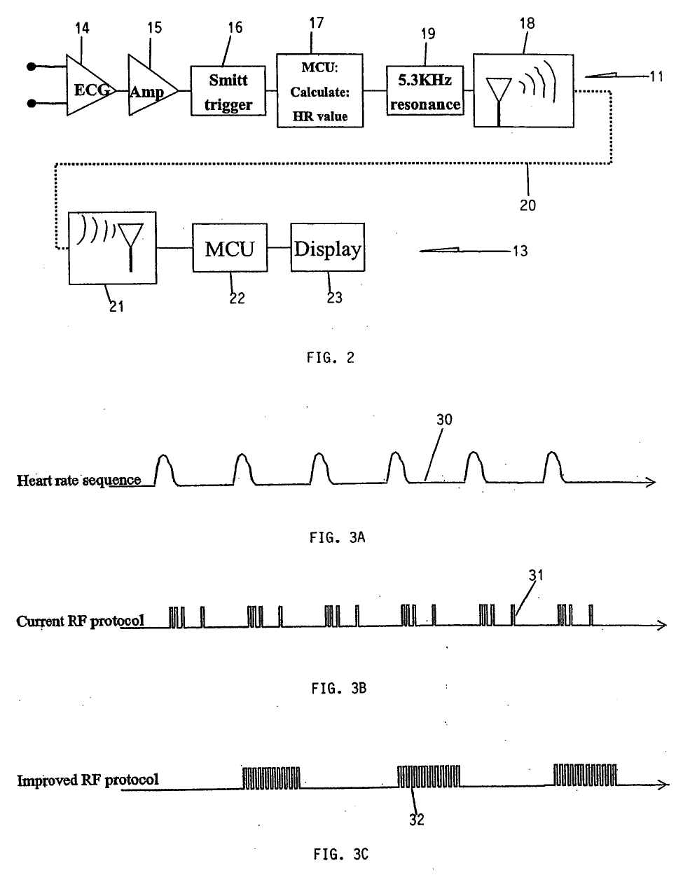

Referring initially to FIGS. 1 and 2 of the drawings, there is shown a wireless heart rate monitoring system 10 embodying the invention, which comprises two separate units i.e. a detection unit 11 and a display unit 13 that are in communication with each other via a wireless link 20 for operation.

The detection unit 11 is in the form of a chest belt, including a strap or straps for attaching around the chest of a user for detection of his/her heartbeat. The display unit 13 resembles a wrist-watch which has an LCD display panel 23 on its casing for displaying a measured heart rate.

The detection unit 11 has a casing 12 and a heart rate detector 14 supported thereby on the rear side for bearing against the user's chest to detect his/her heartbeat. Housed in the casing 12 and connected in series from the detector 14, there are an amplifier 15, a Smitt trigger 16, an MCU (microprocessor control unit) 17, a 5.3 kHz modulator 19 and an RF (radio frequency) transmitter 18.

The detected heartbeat, an ECG signal, will be amplified by the amplifier 15 and then converted by the Smitt trigger 16 into pulses for subsequent processing by the MCU 17. Compared with general triggers, the Smitt trigger 16 offers the advantage of better control over false trigger signals as may be caused by noises.

The MCU 17 is programmed, inter alia, to calculate a heart rate (beats per minute) based on the heartbeat pulses input by the Smitt trigger 16 and then to transform the calculated heart rate into a coded data packet. Besides the calculated heart rate, the MCU 17 includes in the coded data packet a user ID code that actually identifies the detection unit 11, a travelling speed of and/or distance covered by the user also determined by the MCU 17 as auxiliary functions, and error correction bits.

The coded data packet is fed to the transmitter 18, via the modulator 19 for modulation thereby before being transmitted by the transmitter 18 as a wireless RF signal at 5.3 kHz resonance under the control of the microprocessor 17, for reception by the display unit 13.

The display unit 13 includes an RF receiver 21 for receiving the RF signal, i.e. coded data packet, from the detection unit 11, and its own MCU 22 for control including demodulating and processing the coded data packet to extract the encoded data therefrom, i.e. the calculated heart rate and travelling speed/distance if included, for display on the LCD panel 23.

FIG. 3A shows a typical heart rate sequence 30, in that each pulse of the waveform represents a heart beat. A typical RF protocol 31 for a conventional wireless heart rate monitor is shown in FIG. 3B, according to which a burst of pulses is transmitted each time a heartbeat is detected. The heart rate is then calculated at the remote display unit, and displayed. As the bursts of pulses are fairly basic in structure, their transmission is vulnerable to interference by other RF sources, and this sometimes leads to incorrect heart rate readings.

With the use of the subject monitoring system 10, heart rate computation is done right at the detection unit 11, and the calculated heart rate is then transmitted by the detection unit 11 as a coded packet of data (i.e. the improved RF protocol of FIG. 3C) including a user ID, whereby data integrity is ensured.

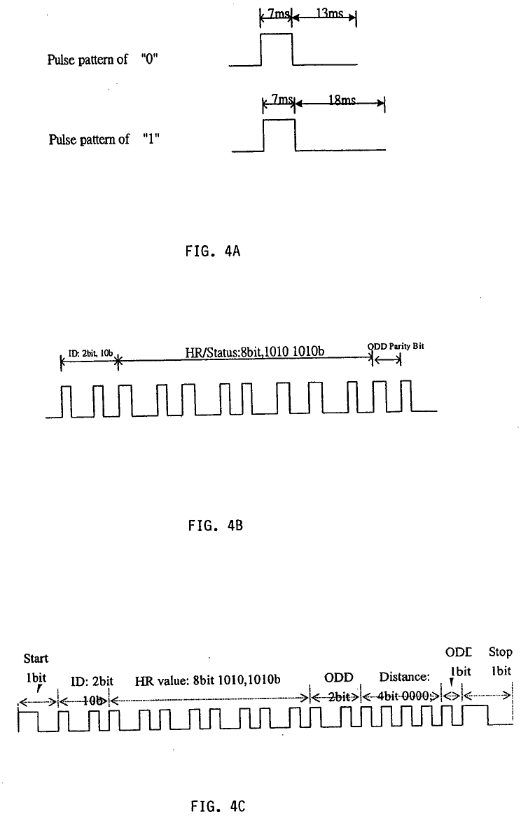

FIG. 4A depicts the pulse patterns of “0” and “1”, which are distinguished by their length. FIG. 4B shows the data format for heart rate information signals generated by the MCU 17 of the detection unit 11 for transmission, which include a 2-bit user ID for user identification (up to four users). Immediately after the 2-bit user ID, there is a 8-bit heart rate reading, which is then followed by a 2-bit ODD parity bit.

FIG. 4C shows the data format of generally the same signals for transmission but extended to carry the heart rate information as well as the speed information. The data string begins with a start bit, followed by the same 2-bit user ID, 8-bit heart rate reading and 2-bit ODD parity bit, and then a 4-bit speed/distance information, a 1-bit ODD parity bit and a stop bit.

The coded data packet contains a calculated heart rate value accompanied by a user ID code, and includes error correction bits. Upon receiving a corrupted packet, the wrist-watch receiver 13 will decode the error correction bits and provide a warning of external interference by flashing an icon, for example, on the display 23. The display 23 may hold the current screen until a correct signal is received again, or if interference persists the receiver 13 may invite the user to change location by, for example, dropping the reading to zero.

Heartbeat signals are gathered and computed by the MCU 17 embedded in the chest belt unit 11 attached to the user, and the computed heart rate in beats per minute is then transmitted to the wrist-mounted unit 13 for display. As the ECG pulse gathering and computation both take place right at the chest belt unit 11 that is in close contact with the user's body (heart), interference is minimised. The computed heart rate from the chest belt unit 11 is transmitted as coded data packets in fixed intervals say every two seconds, with information containing the user ID, heart rate in beats per minute, checksum and error guarding bits to ensure that data is transmitted, received and displayed accurately.

The subject wireless heart rate monitoring system 10 supports multiple user operation to avoid signal interference between users in the same vicinity each using a pair of the detection and display units 11 and 13. This is done by time multiplexing the heart rate transmissions from the individual users in different time slots or channels of a series.

The time slots are preferably equal time periods divided from the aforesaid fixed interval (of say two seconds) between successive data packet transmissions, so that different detection units 11 may transmit data at different times, without crashing of data. To cater for four users, each time slot may be half a second long for a detection unit transmission interval of two seconds.

In each detection unit 11, there is a timing circuit or counter, which is preferably embedded in or implemented by the microprocessor 17, for determining one of the time slots for transmission. This timing circuit is associated with a button (or key or any other form of operator) provided on the front of the detection unit 11 for manual operation to change the transmission time slot.

In practice, upon the wrist-watch receiver 13 receiving a corrupted data packet (by reason of interference) as indicated by the flashing icon, the user should press the said button on the detection unit 11 successively to trigger the timing circuit to switch or shift the time of data transmission to the next time slot until an open or vacant time slot is reached (i.e. the interference problem ceases).

As the heart rate is calculated inside the chest belt unit 11, the remote display unit 13 can be a very simple device for merely receiving and displaying data. With the use of a coherent design on the data packet transmission protocol, the display unit 13 may receive and display data from any other categories of data-transmitting devices such as body fat scales, blood pressure meters and pedometers, etc., whereby a generic health care monitoring system is made possible.

It should be appreciated that modifications of and/or alterations to the described embodiment obvious to persons skilled in the art are not to be considered as beyond the scope of the present invention. For example, a bi-directional wireless communication link may be used between the detection and display units. Furthermore, the display unit may be designed to recognise and receive signals from different heartbeat detection units by other manufacturers found on the market, by including the necessary circuitry for data decoding or conversion, etc.

1. A wireless heart rate monitoring system comprising:
a detection unit and a display unit, the detection and display units being separate and co-operatively in communication via a wireless link, wherein
the detection unit comprises:
a detector casing,
attaching means at the casing for attaching the detector casing to a user,
a heart rate detector at the detector casing for detecting electrocardiogram (ECG) pulses of the user,
a microprocessor in the detector casing for computing a heart rate based on the ECG pulses detected by the heart rate detector, and
a transmitter at the detector casing for transmitting a wireless signal indicative of the heart rate computed by the microprocessor to the display unit; and
the display unit comprises:
a display casing;
a receiver at the display casing for receiving the wireless signal from the detection unit, and
a display at the display casing for displaying the heart rate received by the receiver.

2. The monitoring system as claimed in claim 1, wherein the attaching means comprises at least one strap for attaching around the chest of the user.

3. The monitoring system as claimed in claim 1, wherein the display unit is wrist-mounted.

4. The monitoring system as claimed in claim 1, wherein the transmitter comprises a radio frequency transmitter and the receiver comprises a radio frequency receiver.

5. The monitoring system as claimed in claim 1, wherein the transmitter is controlled by the microprocessor to transmit the wireless signal at substantially fixed intervals.

6. The monitoring system as claimed in claim 1, wherein the detection unit includes timing means for controlling the transmitter to transmit the wireless signal at a different time from that of another detection unit nearby.

7. The monitoring system as claimed in claim 6, wherein the transmitter is controlled by the microprocessor to transmit the wireless signal at substantially fixed intervals, and the timing means comprises a series of time slots divided from the fixed interval, one of the time slots being allocated for the transmitter to transmit the wireless signal.

8. The monitoring system as claimed in claim 7, wherein the detection unit includes an operator, successive depression of which shifts time of transmission of the wireless signal from one time slot to the next vacant time slot.

9. The monitoring system as claimed in claim 1, wherein the wireless signal comprises a coded data packet that includes data representing the heart rate computed by the microprocessor.

10. The monitoring system as claimed in claim 9, wherein the coded data packet further includes a code identifying the detection unit.
